(19) United States
(12) Patent Application Publication
(10) Pub. No.: US 2016/0242689 A1
(43) Pub. Date: Aug. 25, 2016

(54) BLOOD WITHDRAWAL DEVICE

(71) Applicants: Claus-Peter Roehr, Braunschweig (DE); Joachim Vetter, Salzgitter (DE); Bernd Schierwater, Braunschweig (DE)

(72) Inventors: Claus-Peter Roehr, Braunschweig (DE); Joachim Vetter, Salzgitter (DE); Bernd Schierwater, Braunschweig (DE)

(21) Appl. No.: 15/024,439

(22) PCT Filed: Sep. 26, 2014
(86) PCT No.: PCT/EP2014/002615

§ 371 Date: Mar. 24, 2016

(30) Foreign Application Priority Data
Sep. 26, 2013 (DE) 10 2013 219 432.3

(51) Int. Cl. A61B 5/15; A61B 5/151

(52) U.S. Cl. CPC A61B 5/150984; A61B 5/151; A61B 5/150099; A61B 5/150106; A61B 5/150748; A61B 5/150022

(57) ABSTRACT

The invention relates to a blood withdrawal device (10) having an active liquid (12) containing a platelet aggregation inhibitor, a microneedle patch (14) having a plurality of microneedles (20), wherein a plurality of microneedles (20) is configured for dispensing the active liquid (12) into the skin (34) of a patient, and comprising a negative pressure generating device (16) connected to a plurality of microneedles (20) such that blood (46) can be drawn from the skin (34) by means of the microneedles (20). According to the invention the active liquid (12) contains a pain killer, the active liquid (12) is arranged in the blood receptacle (18), and the negative pressure generating device (16) is connected to the blood receptacle (18), such that by generating an excess pressure in the blood receptacle (18), the active liquid (12) can be dispensed by way of microneedles (20), and by generating a negative pressure, blood (46) can be drawn into the blood receptacle (18).

The invention relates to a blood sample collection device in accordance with the generic term of claim 1.

In a multitude of situations it is necessary to withdraw blood from a patient, which can be a human patient or an animal. As a general rule, either a vein is pricked to take a blood sample, or a wound is dealt with a needle, for instance to a fingertip, in such a manner that blood issues from capillaries to the outside and is then collected. This procedure is used by diabetics, for instance, who need to record their blood sugar level at regular intervals.

A disadvantage of taking blood from veins is that it is painful and can only be carried out by medically trained staff in order to prevent major injuries. A disadvantage of pricking a fingertip for example is that only a little amount of blood can be obtained, which can also be contaminated by contact with the skin surface. Another disadvantage is that many people recoil from inflicting themselves a bleeding wound.

A blood sample collection device of the generic kind is known from DE 602 25 859 T2. A disadvantage of the system described there is that taking greater amounts of blood is especially painful.

There is also a blood sample collection device of the generic kind known from WO 02i091 922 A1, which contains a diaphragm pump to suck up blood from out of the skin. This blood sample collection device also has the disadvantage of causing pain.

A device for the release of drugs into the skin and/or for the extraction of liquids from the skin is known from US 2012/0271125 A1. The experience of pain at blood sample collection is reduced by means of an especially high acceleration of the microneedle array. However, the sensation of pain cannot be eliminated completely by this method.

The invention is based on the task of redesigning the taking of blood samples to be less painful.

The invention solves the problem with a blood sample collection device of the generic kind with the characteristics described in claim 1.

One advantage of the invention is that the usage of the microneedle array grants a high probability that at least one microneedle, though especially multiple microneedles, will hit a capillary in the dermis or a blood vessel in the subcutis. For that reason the microneedles can be operated with a small diameter, in a way that the penetration of the subcutis causes only little pain.

As there is a painkiller present in the active liquid, the microneedle patch can be introduced into the skin in such a way that the microneedles initially only penetrate the insensible epidermis. Pain in the lower skin layers is reduced through the release of the painkiller.

According to a preferred design, it is intended to wait until the painkiller has drastically reduced the pain sensitivity and then introduce the microneedles deep enough into the skin that at least one sanguiferous capillary is hit by at least one microneedle. By means of the negative pressure generating device, the blood can be drawn out of the capillary. Therefore, if a painkiller is used, it is possible to carry out a blood sample collection which is at least quasi painless.

The thickness of the epidermis, which is insensible to pain, varies according to age, gender and body region. Therefore, a suitable blood sample collection device must be chosen for each person. For this reason, according to the invention, a kit is planned, which includes two or more blood sample collection devices. The greatest depth of immersion, that is the maximum length that the microneedles surpass the application area, is at least 10% and preferably over 20% greater than that of the blood sample collection device with the lowest depth of immersion. □

Because each microneedle has a very small diameter, it is conceivable that no painkiller be used in particular cases. This can be advantageous when the blood is to be drawn from a relatively insensitive area, e.g. the thigh or the back, for example.

In addition to the painkiller, the active liquid contains a platelet aggregation inhibitor medication. This drug prevents the coagulation of blood so that enough blood can be drawn before the blood sample collection device needs to be removed from the patient. Yet it is conceivable that no antiaggregant be used in some cases. This can occur when the blood is brought into contact with a microchip that carries out the desired analysis immediately after extraction from the body and before the blood turns unanalyzable by clotting, for example.

It is especially favorable, that both a platelet aggregation inhibitor and an analgesic medication are present in the active liquid. In this case, there is no time pressure for the extraction of blood because of the antiaggregant, allowing the extraction to wait until the painkiller has reduced sensitivity to a large extent.

This enables the extraction of a sufficient amount of blood with little or even no pain for the patient.

In the context of the description at hand, a blood sample collection device is defined as a device developed to extract blood. The device generating negative pressure and the microneedle array are at least indirectly connected in such a way that the blood sample collection device can be held and applied with one hand.

The active liquid is defined as a liquid that is present within the blood sample collection device before blood is drawn from a patient using the blood sample collection device. The active liquid is part of the blood sample collection device.

A painkiller is defined as a chemical substance that temporarily completely blocks or at least lessens pain sensitivity. For example, Mepivacain or Meivacain are considered as analgesic.

The analgesic can be a local anesthetic, for example an aminoamide, aminoester, or analgesic.

A microneedle array is defined as an arrangement set of microneedles arranged in a dense manner that can be introduced into the skin of a patient jointly, e.g. by moving the blood sample collection device with one hand.

For example, the microneedle array is developed in such a way that it can be surrounded by an imaginary circle with a diameter of 10 cm, especially of 5 cm, but preferably of 3 cm.

The microneedle array encompasses at least 10 but preferably 25 or more microneedles. Thus it is ensured that with a high probability at least one microneedle hits a sanguiferous capillary from which blood can be extracted.

A microneedle is understood to be a needle whose outer diameter measures no more than 200 μm, and preferably less than 100 μm. The microneedles are manufactured out of a material which is sufficiently ductile so as to prevent breaking.

The negative pressure generating device is defined as a device that automatically generates and/or applies a negative pressure upon the microneedle array when activated and requires no further action to maintain a negative pressure after activation. Preferably this is achieved by the mechanics of passive components that require no chemical or electrical energy, ensuring that the negative pressure generating device is free of rotating components.

Preferentially, the active liquid is stored in a receptacle that is airtight. It is then possible to inject the active liquid through the microneedles into the skin of the patient by applying positive pressure onto the receptacle without risk of air getting into the patient's body. Naturally it is possible that little amounts of air will be present in the receptacle but it is important that these amounts of air remain so little that they pose no threat to the patient and do not compromise the taking of a blood sample.

The blood sample collection device contains a blood receptacle which is connected to a plurality of microneedles in such a way that blood can be drawn through the microneedles into the receptacle. It is possible, but not necessary, that all microneedles of the microneedle patch are connected to the blood receptacle. It is also conceivable that the microneedles are connected to the reservoir for active liquid.

It is then possible to inject the active liquid through the microneedles into the skin.□

Moreover, it is possible in this case to use needles with a greater outer diameter, which are connected to the blood receptacle, in addition to the usage of the microneedles. In this case, the painkiller can be injected into the skin through the microneedles first. As soon as the painkiller takes effect, the needles with the greater diameter can be introduced into the skin with no pain. A blood sample collection device of this kind has the advantage of having a greater stream of blood flow through the blood sample collection needles, that is, those needles through which blood can be drawn into the blood receptacle. This way an especially high amount of blood can be taken or a predetermined amount of blood can be taken in a shorter amount of time. □

Though it has to be taken into account that a heightened pain sensitivity occurs after the collection of a blood sample taken in this way after the effect of the painkiller has ceased (when compared to the exclusive usage of microneedles), it can be appropriate to accept this disadvantage, namely when the advantages of taking more blood or in a shorter amount of time prevail.□□

The active liquid is stored in the blood receptacle. Thus it is possible that first the active liquid is injected into the patient's skin through the microneedles and that then the blood is drawn into the blood receptacle through the same microneedles. Such a blood sample collection device is of particularly simple construction. It is further convenient when the negative pressure generating device is connected to the blood receptacle, so that the active liquid can be ejected through the microneedles by generating a positive pressure in the active liquid reservoir, and that blood can be drawn into the blood receptacle by generating a negative pressure in the blood receptacle.

It is favorable for the negative pressure generating device to contain a switch for activating the device's negative pressure. For example, it is possible that the switch is built to destroy a partition between a vacuum container and the blood receptacle. Alternatively or additionally, it is possible that the negative pressure activation switch holds a spring in place under tension, in which at least partial destruction of the switch will release the spring and the vacuum will be generated.

It is preferred that the blood sample collection device contains a surface for applying pressure, acting as a button, to eject the active liquid from the active liquid reservoir through the microneedles. It is particularly convenient when this button area is constructed in a way that allows activation with one finger or multiple fingers.□

It is especially favorable when the switch is built in such a way that the negative pressure generating device is activated when the button area is pressed, in particular in direction of the patient's skin. In this case, it is sufficient to press the button area in order to first eject the active liquid into the patient's skin and then to activate the negative pressure generating device. After a single push onto the button area, the blood sample collection device can remain on the skin of the patient, where it automatically takes blood, without requiring any action.

Corresponding to a preferred construction, the blood sample collection device comprises a mounting compound, which allows for temporary fixation on the patient's skin.

For example the mounting compound comprises an adherent compound, by which the blood sample collection device can be glued onto the skin, especially in a hermetically sealed fashion. This feature allows to keep the blood sample collection device affixed to the skin of the patient via negative pressure. As such, the adherent compound can be developed with a comparably weak glue and with a comparably small adherent area, because the negative pressure facilitates fixation to the skin. Yet it is also possible that the blood sample collection device is fixable to the skin of the patient using a mounting device without application of negative pressure.

It is convenient when the adherent compound comprises a foam, which is preferably not resilient, meaning that it will recover no more than 70% of its former thickness within 30 seconds after squeezing, also without continuous application of force. This way the microneedles can be introduced into the skin without the need for a great force to keep the blood sample collection device in place.

It is possible that the foam contains glue, located in pores of the foam. It is convenient when the adherent compound is capped with a removable cover.

Preferably the blood sample collection device features a contact surface for application onto the patient's skin and a compression compound. This compound can be brought from an uncompressed state into a compressed state by pressure. The microneedles surpass the contact surface in particular by 1.0 mm, but no less than 0.7 mm, when the compression compound is in compressed state.

It is convenient when the microneedles do not surpass the contact surface when the compression compound is in uncompressed state. In this case the microneedles are not or barely visible when the blood sample collection device is applied. In accordance to a preferred construction, the compression compound can be formed of foam rubber, preferably of the same type found within the adherent compound.

When the microneedles stand out at least 0.7 mm over the compression element, that is when the immersion depth measures at least 0.7 mm, blood can be taken from an infant or a sixty-year-old from blood vessels belonging to the subcutaneous tissue of different body parts, for example from the palm of the hand or fingertip. The immersion depth should be deeper than 0.7 mm for a physically laboring man e.g. 1.0 mm, allowing for blood to be drawn from various parts of the patient.

The contact surface is defined as the area of the blood collection device that comes into contact with the surface of the patient's skin when no negative pressure is applied. The contact surface can be the surface of the adherent compound, for example.

Alternatively or additionally, the mounting compound can encompass a cuff by which the blood sample collection device can be fixed to a patient with form-locking. This can be an inflatable cuff, allowing for the patient's blood pressure to be measured.

It is convenient when the microneedles are so long that they can be introduced at least 1.5 mm and preferably over 2 mm into the skin when the blood sample collection device is mounted and pressure is applied onto the button area.

The dermis is ca. 1.5 mm thick, so that at introduction of over 1.5 mm, a sanguiferous capillary or blood vessel is hit with a high probability.

According to a preferred construction, the adherent compound surrounds a suction area and the blood sample collection device contains a negative pressure chamber which is opened toward the suction area, so that the blood sample collection device can be sucked to the patient's skin by means of the negative pressure present in the negative pressure chamber.

The negative pressure chamber can feature a mechanical spring, for example, which is held in a position of tension by a switch. The switch releases the spring when pressure is applied to the button area.

Yet it is also possible that the switch is activated in another way. The negative pressure chamber is dispensable if the mounting compound contains a cuff, because the blood sample collection device can then be fixed to the patient's body using the cuff.

It is convenient when the microneedle array is manufactured so that it is removable from the active liquid reservoir. In this case it is possible to remove the microneedle array after the taking of a blood sample so that any hazard can be avoided.

Preferably, the microneedle array can be clicked into, glued onto, or connected to the active liquid reservoir by perforation and can be removed without tools or cutting. It is possible, but not necessary, that the microneedle array is directly linked to the active liquid reservoir. An indirect link is also possible.

As per a preferred design, a microchip is located within the blood receptacle, allowing for testing of at least one blood parameter quantitatively and/or qualitatively.

In particular the microchip can be built to carry out at least one genetic diagnosis.

The microchip preferably contains an energy converter unit converting radiant energy into electric energy. For example, the energy converter unit contains an electromagnetic coil, so that application of an external time-varying magnetic field can induce an electric current in the microchip. It is also possible that the microchip can be read out by radiation with light.

Preferably the mounting compound contains an expandable, especially inflatable cuff. This can be connected to the blood sample collection device in a way that an expansion of the cuff applies pressure to the blood sample collection device and thereby allows for passing the active liquid into the microneedle array. In other words the active liquid can be injected into the patient's skin by inflating the cuff. For instance, by reducing the pressure inside the cuff, it is then possible to draw blood into the blood receptacle by means of the negative pressure generating device.

According to a preferred construction, the blood sample collection device contains a pressure generating device that is connected to the cuff in order to apply a positive pressure. An electronic control unit is designed to carry out the following steps:

(i) Actuate the pressure generating device, so that it exposes the cuff with a release pressure, selected to release the active liquid through the microneedles.

(ii) Actuate the pressure generating device to expose the cuff with an extraction pressure selected to draw up blood via the microneedles.

In this case the blood sample collection device solely needs to be placed correctly on the patient's body, for example on a limb. The electronic control unit then carries out all steps necessary to extract blood from the patient automatically, making it self-actualizing. After blood withdrawal, merely the cuff needs to be taken off, and the blood sample is taken. Specialized medical staff is not required.

It is especially favored that the control unit is set to additionally execute the step of actuating the pressure generating device, so that it exposes the cuff with a starting pressure selected to bring the compression compound into compressed state. This is especially advantageous when the microneedles do not surpass the compression compound in uncompressed state. In other words the blood sample collection device can be placed without the patient seeing or feeling the needles. By application of the different aforementioned pressures, blood can then be taken with little or no pain.

According to a preferred construction the active liquid contains at least one vasodilatory substance, for example a beta blocker or a calcium channel antagonist.

In accordance to a preferred design the active liquid contains at least one anticoagulant. An anticoagulant is understood to be a substance that inhibits clotting of the blood. It can be a direct or indirect anticoagulant. Examples include hirudin, apixaban, dabigatran, rivaroxaban as well as vitamin K antagonists and heparins.

So, in particular the active liquid contains an anticoagulant, a platelet aggregation inhibitor, a vasodilator and a local anesthetic or analgesic.

In the following the invention is further explained on the basis of the attached drawings.

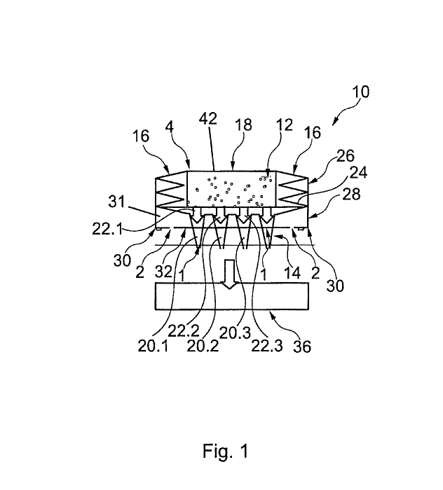

FIG. 1 Schematic cross-section of a blood sample collection device according to the invention

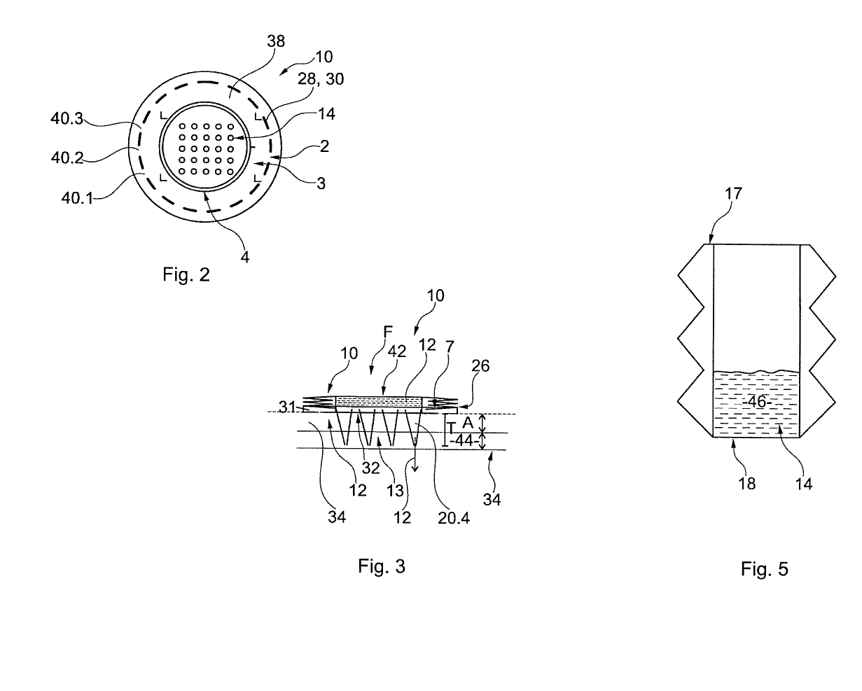

FIG. 2 View from below of the blood sample collection device according to FIG. 1

FIG. 3 Blood sample collection device after ejection of the active liquid

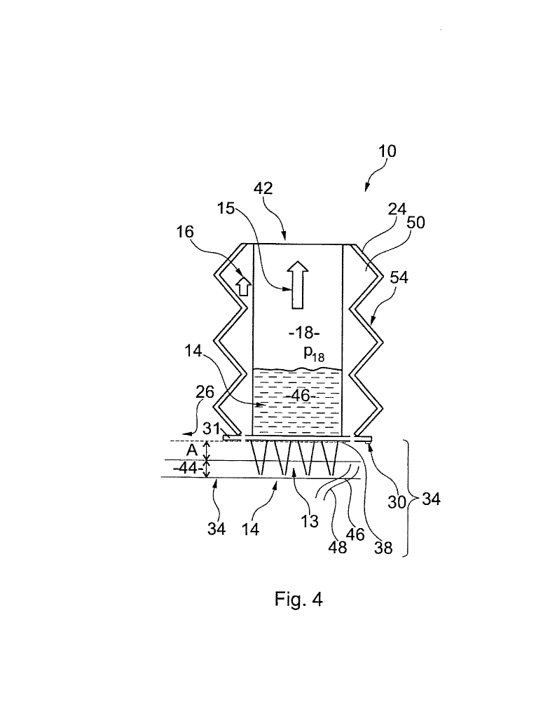

FIG. 4 Blood sample collection device while taking a blood sample

FIG. 5 Blood sample collection device after taking the blood sample

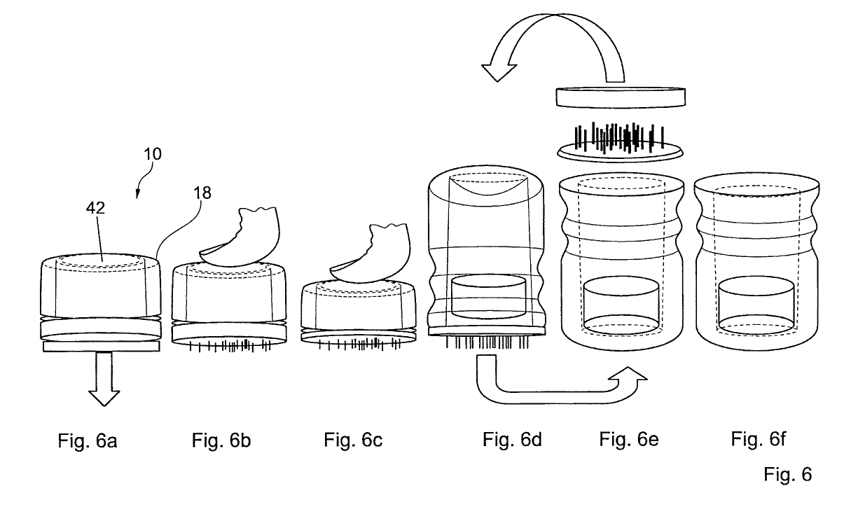

FIG. 6 Schematic depiction of the taking of blood using the blood sample collection device from 6a to 6f

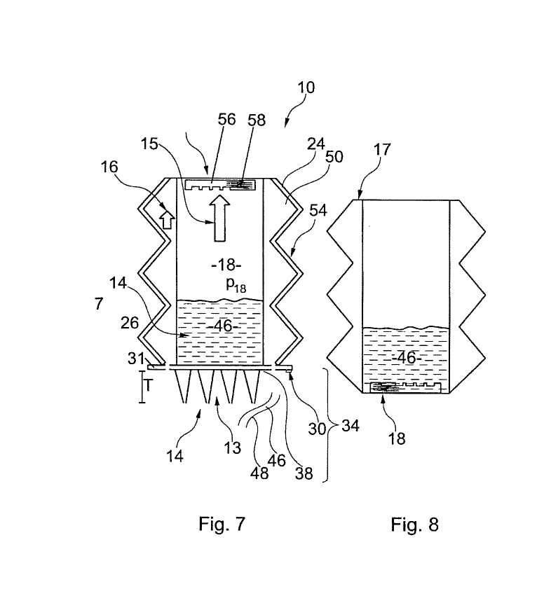

FIG. 7 Schematic cross-section of a blood sample collection device according to a second design of the invention after drawing in the blood

FIG. 8 Blood sample collection device according to FIG. 7 with detached microneedle array

FIG. 1 shows a blood sample collection device 10 according to the invention, that comprises an active liquid 12, a microneedle array 14 and a negative pressure generating device 16. The active liquid 12 has a volume V₁₂ of for instance less than 75 microliters, especially less than 50 microliters. In the case at hand, there are 25 microliters present.

The active liquid contains an anticoagulant, for example in form of heparin, and/or an analgesic, for instance 1% mepivacaine or rather Carbocaine. However, it is also possible to use other local anesthetics.

The active liquid is stored in a reservoir 18, that also functions as a blood receptacle in the case at hand. However, it is also possible that the active liquid 12 is stored in a separate active liquid reservoir, that is not concurrently a blood receptacle.

The microneedle array 14 contains a plurality of microneedles 20.1, 20.2, . . . , that are in connection with the blood receptacle 18 via canals 22.1, 22.2, . . . , in the case at hand. Altogether there are 25 microneedles present in the construction at hand, yet it is also possible that more or less microneedles are used.

The negative pressure generating device 16 contains at least one spring 24, that is for example made of plastic or metal. The spring 24 is held in a position of tension by an activation device 26 to prevent elongation.

The blood sample collection device 10 contains a mounting compound 28, that comprises an adherent compound 30, in the construction at hand. In the case at hand the adherent compound is a compression compound 31 in form of a foam compound, that is at least partially infused with glue. The compression compound 31 is at least partially also arranged around the microneedle array 14.

There is a contact surface 32 at the mounting compound 28, which is located at the side facing away from the receptacle 18, in the case at hand. The blood sample collection device 10 is brought into contact with the patient's skin 34 via the contact surface 32 in case of application.

FIG. 1 also show a protective cover 36 that is constructed in such a way that the microneedle array 14 is not exposed when the protective cover 36 is put on.

FIG. 1 further shows that the blood sample collection device 10 contains a button area 42, that is placed on the receptacle 18 in the construction at hand.

FIG. 2 shows a view from underneath the blood sample collection device 10. It is evident that the adherent compound 30 is formed in a ring shape. The adherent compound 30 comprises a suction area 38, in which a negative pressure can be created when exercising the blood sample collection device, as described below. It is possible that the adherent compound 30 shows interruptions 40.1, 40.2, . . . as shown in FIG. 2. This allows for a low-volume airflow into the suction area 38.

FIG. 2 also shows that little amounts of active liquid have remained in the active liquid reservoir 18. Blood entering the active liquid reservoir 18 in later steps (as described in the following) is hindered from clotting by the heparin in the active liquid 12.

FIG. 3 shows the state after a force F has been exerted onto the button area 42, so that the active liquid 12 has been injected into a subcutis 44 of the skin 34 via the microneedles 20 (reference numerals without counting suffix (i.e. 20.1, 20.2) refer to all relevant elements). When the force F was exerted onto the button area 42, the activation device 26 has been activated by breaking it. For this purpose the activation device 26 can have a non-illustrated predetermined breaking point.

FIG. 3 also illustrates that the microneedles 20 of the microneedle array 14 surpass the contact surface 32 with an immersion depth T when a force F exerted onto the button area 42 is so great that the active liquid 12 is injected in relevant amounts for anesthetization. The immersion depth ranges from 0.6 to 1.5 millimeters and depends on the pliability of the compression compound 31.

FIG. 4 shows the state in which the spring 24 springs back from its position of tension into its relaxed position. This causes a negative pressure p₁₈ to be applied to the blood receptacle 18. By means of this negative pressure, blood 46 is taken out of a capillary 48 (as schematically drawn) into the blood receptacle 18.

By expansion of the spring 24, which can be a coil spring for example, also a negative pressure p₅₀ is created in a negative pressure chamber 50. The negative pressure chamber 50 is connected to the suction area 38 via an opening 52. This causes the blood sample collection device 10 to be sucked onto the skin 34 and connected firmly to it. It can be advantageous to provide for a valve or nozzle 54, so that air streams into the negative pressure chamber 50. This ensures that the button area 42 further moves away from the skin 34, so that the negative pressure p₁₈ in the blood receptacle 18 is maintained.

FIG. 5 shows the state in which the microneedle array 14 has been removed (compare with FIG. 4). The blood 46 is now located in the blood receptacle 18 and can be analyzed.

FIG. 6 shows the functioning of the blood sample collection device 10 with another example in parts 6a to 6f. It can be seen that the blood receptacle 18 is a flexible plastic container.

FIG. 7 shows an alternative construction that comprises a microchip 56. This can be located on the side of the blood receptacle 18 opposite to the microneedle array 14. As shown in FIG. 8, the blood can be brought into contact with the microchip 56 by turning around the blood receptacle 18. The microchip 56 is supplied with energy via the induction coil 58 by applying an external alternating field.

List of Reference Numbers

| | |
|---|---|
| 10 | Blood sample collection device |
| 12 | Active liquid |
| 14 | Microneedle array |
| 16 | Negative pressure generating device |
| 18 | Container: Active liquid reservoir, blood receptacle |
| 20 | Microneedle |
| 22 | Canal |
| 24 | Spring |
| 26 | Switch |
| 28 | Mounting device |
| 30 | Adherent compound |
| 31 | Compression compound |
| 32 | Contact surface |
| 34 | Skin |
| 36 | Protective cover |
| 38 | Suction area |
| 40 | Interruption |
| 42 | Button area |
| 44 | Subcutis |
| 46 | Blood |
| 48 | Capillary |
| 50 | Negative pressure chamber |
| 52 | Opening |
| 54 | Valve |
| 56 | Microchip |
| 58 | Induction coil |
| F | Force |
| P₁₈ | Pressure |
| P₅₀ | Pressure |
| T | Immersion depth |
| V₁₂ | Volume |

1. Blood sample collection device, comprising:
a) an active liquid that contains a platelet aggregation inhibitor,
b) a microneedle array, that includes a plurality of microneedles,
c) wherein the plurality of microneedles is configured for injection of the active liquid into skin of a patient, and
d) a negative pressure generating device connected to the plurality of microneedles so that blood can be drawn out of the skin via the microneedles;
wherein
e) the active liquid contains a painkiller,
f) the active liquid is located within a blood receptacle, and
g) the negative pressure generating device is connected with the blood receptacle in a way that allows injecting the active liquid through the plurality of microneedles by generating a positive pressure in the blood receptacle and to suck up blood into the blood receptacle by generating a negative pressure.

2. The blood sample collection device according to claim 1 wherein the negative pressure generating device possesses a switch that can activate the negative pressure generating device to generate a negative pressure.

3. The blood sample collection device according to claim 2 further comprising
a button area located so that the active liquid can be put under pressure (p₁₈) by exerting a force (F) onto the button area, and wherein the switch is located so that the negative pressure generating device can be activated by exerting the force (F) onto the button area.

4. The blood sample collection device according to claim 1 further comprising a mounting device which allows the blood sample collection device to be temporarily fixed to the skin of a patient.

5. The blood sample collection device according to claim 1 further comprising
a contact surface for placing the blood sample collection device against the skin of a patient, and
a compression compound, that can be brought from an uncompressed state into a compressed state by application of pressure (p₁₈),
wherein the plurality of microneedles surpass the contact surface by at least 1 millimeter when the compression compound is in compressed state.

6. The blood Blood sample collection device according to claim 5 wherein
the plurality of microneedles do not surpass the contact surface when the compression compound is in an uncompressed state.

7. The blood sample collection device according to claim 4
wherein the mounting device comprises a cuff with which the blood sample collection device can be fixed to the patient's skin with form-locking.

8. The blood sample collection device according to claim 1 wherein the plurality of microneedles are formed with a length that they penetrate the patient's skin by at least 0.7 millimeters when the blood sample collection device is collocated on the skin of a patient and a pressure (p₁₈) is applied onto a pressure patch or button area.

9. The blood sample collection device according to claim 1 further comprising
an adherent compound surrounding a suction area, and
a blood sample collection device containing a negative pressure chamber that is opened towards the suction area so that the blood sample collection device can be attached to the patient's skin using the suction caused by negative pressure in the negative pressure chamber.

10. The blood sample collection device according to claim 1 further comprising
a mounting device containing an expandable, inflatable cuff, and
a blood sample collection device located in such a way in relation to the inflatable cuff that a pressure (p₁₈) can be exerted onto the blood sample collection device by expansion of the cuff, so that the active liquid can be injected through the microneedle array, where
the blood sample collection device contains
a pressure generating device that is connected to the inflatable a cuff for application of a positive pressure, and
an electronic control unit designed to automatically carry out an operation including the following steps:
actuation of the pressure generating device, so that it charges the inflatable cuff with an active compound release pressure (p₁₈), selected to release the active liquid through the plurality of microneedles, and
actuation of the pressure generating device, so that it charges the inflatable cuff with an extraction pressure, selected to suck up blood via the plurality of microneedles.

11. The blood sample collection device according to claim 1 wherein the active liquid contains a vasodilatory compound.

12. The blood sample collection device according to claim 1 wherein the active liquid contains at least one anticoagulant.
